(19) United States
(12) Patent Application Publication
(10) Pub. No.: US 2026/0183213 A1
(43) Pub. Date: Jul. 2, 2026

(54) Stable And Mild Surfactant Wash Composition

(71) Applicant: Conopco, Inc.,d/b/a UNILEVER, Englewood Cliffs, NJ (US)

(72) Inventors: Haley Ruth SHELLEY, New Haven, CT (US); Douglas John Hiban, Newtown, CT (US); Melissa Ann Maffeo, Southington, CT (US); Edward Joe Martinez, New Haven, CT (US)

(73) Assignee: Conopco, Inc., d/b/a UNILEVER, Englewood Cliffs, NJ (US)

(21) Appl. No.: 19/005,027

(22) Filed: Dec. 30, 2024

(51) Int. Cl. A61K 8/64; A61K 8/365; A61K 8/368; A61K 8/42; A61K 8/44; A61K 8/46; A61Q 5/02; A61Q 19/10

(52) U.S. Cl. CPC A61K 8/64; A61K 8/365; A61K 8/368; A61K 8/42; A61K 8/442; A61K 8/466; A61Q 5/02; A61Q 19/10; A61K 2800/33; A61K 2800/34; A61K 2800/596

(57) ABSTRACT

The present invention is directed to an isotropic wash composition. The composition is stable, comprises alfa olefin sulfonate and a co-anionic surfactant. The composition comprises an amphoteric surfactant, low salt content and can comprise a peroxisome proliferator-activated receptor (PPAR) agonist.

### FIELD OF THE INVENTION

The present invention is directed to a stable and mild surfactant wash composition. More particularly, the composition comprises a C₁₀₋₂₀ alpha olefin sulfonate, co-anionic surfactant, and a nonionic surfactant, a fatty acid, fatty alcohol, fatty amide or a mixture thereof. The wash composition comprises less than 16% by weight total surfactant, maintains excellent washing and foaming characteristics, and surprisingly, maintains stable viscosity with low salt and when total anionic surfactant in the composition is at least 35% by weight C₁₀₋₂₀ alpha olefin sulfonate. Such a wash composition can be formulated substantially free of at least one of a sulfate-based surfactant, paraben, hydantoin, isothiazolinone, phthalate, soap, dioxane and silicone oil.

### BACKGROUND OF THE INVENTION

Aqueous wash compositions, such as detergents, face washes, shampoos and body and hand washes are common and enjoyed by many consumers. Such compositions typically have water as the predominant ingredient, and they are often sold in plastic bottles, sachets or tubes. The compositions are conventionally formulated to have a viscosity that is customary for consumer use and easy for evacuation from the package they are sold in.

While consumers continue to want products with traditional wash composition characteristics, many are demanding compositions that are mild to their skin and hair. Notwithstanding, wash compositions with mild anionic surfactants are known to present challenges when stabilizing product viscosity. In wash compositions that are, for example, deplete of sulfate-derived surfactants, including ethoxylated surfactants like sodium lauryl ether sulfate, it is harder to maintain consistent composition viscosities during production, typically resulting in costly rework after product is made. Often, the solution to viscosity challenges is achieved with the use of high levels of salt and thickener or a combination of the same. While such components can, for example, aid in viscosity stabilization, they can be unreliable when product is subject to temperature fluctuations and certain consumers do feel high levels of thickeners impede product foam richness they often desire. Salts, especially at levels that exceed 1.65% by weight by of a wash composition, have the potential to dry out and irritate the skin of a subset of consumers.

It is of increasing interest to develop a wash composition that is mild and maintains a consumer desirable viscosity. The present invention, therefore, is directed to a wash composition comprising a C₁₀₋₂₀ alpha olefin sulfonate, a co-anionic surfactant, and a nonionic surfactant, fatty acid, fatty alcohol, fatty amide or a mixture thereof. The wash composition comprises less than 16% by weight total surfactant, maintains excellent washing and foaming characteristics, and surprisingly, maintains stable viscosity with low or no salt even when total anionic surfactant in the composition is at least 35% by weight C₁₀₋₂₀ alpha olefin sulfonate. Moreover, the wash composition of the present invention can be formulated substantially free of at least one of a sulfate-based surfactant, paraben, hydantoin, isothiazolinone, phthalate, soap, dioxane and silicone oil.

### Additional Information

Efforts have been disclosed for making wash compositions. In U.S. Pat. No. 4,514,444, cleaning and conditioning compositions having improved stability are described.

Other efforts have been disclosed for making wash compositions. In U.S. Patent Application Publication No. 2023/0414468 A1, a wash composition that does not irritate the eyes and that has anionic surfactant, amphoteric or zwitterionic surfactant or both, and nonionic surfactant or cationic polymer or a mixture thereof is described.

Further efforts have been disclosed for making wash compositions. In Australian Application No. 2018201747 A1, a composition that includes alpha olefin sulfonate, EDTA and sodium dodecyl sulfate is described.

Even other efforts have been disclosed for making wash compositions. In European Patent No. 4 204 531 B1, a detergent composition with secondary alkane sulfonate and a hydroxy sultaine is described.

None of the additional information describes a wash composition that comprises an alpha olefin sulfonate as claimed herein.

### SUMMARY OF THE INVENTION

In a first aspect the present invention is directed to a wash composition comprising:
a. from 1.2 to 4%, and preferably, from 1.5 to 3.5%, and most preferably, from 1.5 to 3% or from 1.6 to 2.6% or from 1.6 to 2.5% or from 1.7 to 2.2% by weight of C₁₀₋₂₀ alpha olefin sulfonate;
b. from 1.2 to 4%, and preferably, from 1.5 to 3.5%, and most preferably, from 1.5 to 3% or from 1.6 to 2.6% or from 1.6 to 2.5% or from 1.7 to 2.2% by weight of a co-anionic surfactant;
c. a component comprising from 0.28 to 1.5%, and preferably, from 0.3 to 1.2%, and most preferably, from 0.3 to 1% or from 0.35 to 0.9% or from 0.38 to 0.8% or from 0.44 to 0.7% by weight of a nonionic surfactant, a C₁₀₋₂₀ fatty acid, C₁₀₋₂₀ fatty alcohol, C₁₀₋₂₀ fatty amide or a mixture thereof, with the proviso that if the C₁₀₋₂₀ fatty acid is included in the component, the fatty acid makes up from 25 to 50%, or from 30 to 45% or from 30 to 42% or from 30 to 40% by weight of the component;
d. from 3 to 10%, and preferably, from 4 to 8%, and most preferably, from 5 to 7.5% or from 5.5 to 7.5% or from 6 to 7.5% or from 6.5 to 7.25% or from 6.7 to 7.2% by weight amphoteric surfactant;
e. optionally, from 0.001 to 1%, and preferably, from 0.03 to 0.8%, and most preferably, from 0.03 to 0.7% or from 0.035 to 0.6% or from 0.04 to 0.1% or from 0.04 to 0.09% or from 0.04 to 0.07% by weight of a peroxisome proliferator-activated receptor (PPAR) agonist, the PPAR agonist comprising at least 65%, and preferably, at least 75%, and most preferably, from 80 to 100% or from 85 to 100% or from 90 to 100% or from 60 to 90% or from 90 to 98% or 100% by weight 12-hydroxystearic acid based on total weight of the PPAR agonist;
f. less than 1.65%, and preferably, less than 1.6%, and most preferably, from 0.05 to 1.55% or from 0.5 to 1.55% or from 1 to 1.5% or from 1.1 to 1.45% by weight salt;
g. from 68 to 90%, and preferably, from 72 to 88%, and most preferably, from 75 to 86% or from 78 to 85% by weight water,
   wherein:
i) the composition is substantially free of sulfate-based surfactant;
ii) the composition is isotropic;
iii) the C₁₀₋₂₀ alpha olefin sulfonate makes up from 35 to 75%, and preferably, 40 to 70%, and most preferably, from 40 to 65% or from 45 to 60%, or from 45 to 55% by weight of the total weight of the anionic surfactant in the wash composition; and
iv) the composition has a pH from 5.7 to 8, and preferably, from 6 to 7.5, and most preferably, from 6.1 to 7.2 or from 6.1 to 6.9 or from 6.2 to 6.8.

In a second aspect, the present invention is directed to the wash composition of the first aspect of the invention where the mixture defined in paragraph c is 85 to 100% or from 90 to 100%, or from 92 to 98% or from 93 to 97% or from 95 to 100% or 100% nonionic surfactant and fatty acid.

In a third aspect, the invention is directed to the use of a nonionic surfactant and fatty acid in combination with a C₁₀₋₂₀ alpha olefin sulfonate to produce a stable wash composition substantially free of a sulfate-based surfactant.

In a fourth aspect, the present invention is directed to a method for washing a surface, including skin or hair, with the wash composition according to the first aspect of the invention.

All other aspects of the present invention will more readily become apparent from the description and Examples which follow.

Skin, as used herein, is meant to include skin on the arms (including underarms), face, back, feet, neck, chest, hands, legs, buttocks and scalp. Stable means the wash composition displays no syneresis and does not have a discernible viscosity change after being stored for three (3) weeks, and preferably, for one (1) month, and most preferably, for two (2) months at 22° C. as determined by trained panelists. Not having a discernible viscosity change means the wash composition displays less than a 36%, and preferably, less than a 34.5%, and most preferably, less than a 33% viscosity change at the defined times and temperatures described and even when formulated with less than 16% by weight total surfactant and less than 1.65% by weight salt where viscosity is measured at 25° C. with a Discovery HR-2 Rheometer using sand blasted plates having a 1000-micron gap and a shear rate of 10-1 s⁻¹. Stable, as it relates to the composition of the present invention, also means that should such a composition having a total weight percent of X total solids (e.g., surfactant) be reformulated with 25% less total solids (i.e., 0.75X to account for or mimic potential ambiguities in product supply), the viscosity difference between the compositions would be less than 36%. Excellent washing and foaming characteristics means a foam volume and wash off characteristics consistent with traditional isotropic washes having about 2 to 2.5% by weight sodium lauryl sulfate as the anionic surfactant and as determined by skilled panelists washing with water at about 37° C. and wash composition and water at a weight ratio from 1:6 to 1:3 or from 1:5 to 1:4.5 or from 1:4.5 to 1:4.

Isotropic means a composition with micelles arranged without directional preference. The wash composition of the present invention is isotropic. Soap, as used herein, means salt of a fatty acid. Zwitterionic means being dipolar, a surfactant having charged groups of opposite signs where the positive charge is typically an ammonium moiety and the negative charge is typically a carboxylate moiety as often displayed by amino acids. Amphoteric surfactant means a surfactant that changes from positive or cationic to negative or anionic as the pH goes from acidic to basic. For the avoidance of doubt, use of the term amphoteric is meant to include zwitterionic surfactants.

The wash composition of the present invention is one which typically has a viscosity of at least 100 mPa-s where viscosity is taken as previously defined. The wash composition of this invention is one suitable to be wiped or washed off, and preferably, washed off with water. Such a composition can be a home care cleaning composition (e.g., for tabletops, glass, toilets, dishes, upholstery, or laundry) but is preferably a shampoo, make-up wash, facial wash, hand wash, or personal care and liquid body wash. In an embodiment of the invention, the wash composition can have a viscosity from 500 to 18,000 mPa-s, and most preferably, from 1,000 to 16,000 mPa-s (or 1,200 to 15,000 mPa-s or 1,500 to 14,000 mPa-s or 1,800 to 13,500 mPa-s or 2,000 to 13,000 mPa-s or from 3,000 to 10,000 mPa-s or from 3,500 to 9,500 mPa-s). When used for hand applications, the wash composition of the present invention will preferably have a viscosity that is at least 500 mPa-s and not higher than 6,000 mPa-s.

The wash composition of this invention may, optionally, comprise medicinal or therapeutic agents, but preferably, is a wash which is for cosmetic and non-therapeutic uses to remove dirt, oil, make-up or the like from surfaces, especially the skin and scalp. In a preferred embodiment, the wash composition is a personal wash composition, and therefore, a liquid face or body wash for use while showering or bathing. The wash composition of the present invention may optionally comprise skin benefit ingredients added thereto such as emollients, vitamins and/or derivatives thereof, resorcinols, retinoic acid precursors, colorants, moisturizers, sunscreens, antibacterial agents, ceramides, mixtures thereof or the like. Such skin benefit ingredients (or agents) can be water and/or oil soluble. If used, oil soluble skin benefit agents typically make up to 2% (or up to 1.5%) by weight of the wash composition with the proviso that PPAR agonist, if used, makes up from 0.001 to 1% by weight of the wash composition. Water-soluble skin benefit agents, when optionally used, may make up to 10% (or up to 5%) by weight of the wash composition. The wash composition will typically have a pH from 5.7 to 8, and preferably, from 6 to 7.5, and most preferably, from 6.1 to 7.2 or from 6.1 to 6.9 or from 6.2 to 6.8. PPAR agonist means compounds that activate receptors identified as peroxisome-proliferator-activated receptors, a lipid that activates the nuclear receptor PPAR, and particularly, alpha.

As used herein, “substantially free of” as it relates to sulfate-based surfactants (e.g., sodium lauryl sulfate or sodium pareth sulfate) means less than 1%, and preferably, less than 0.8%, and most preferably, less than 0.5%, even more preferably, less than 0.2% or less than 0.1% by weight of the wash composition. In an embodiment of the invention, “substantially free of sulfate-based surfactant includes a wash composition with 0.0% (i.e., none) by weight sulfate-based surfactant. Regarding the non-sulfate-based ingredients (i.e., ingredients, like a hydantoin preservative, phthalate, polymeric thickener, polymeric suspending agent), substantially free of means less than 0.6% by weight, and preferably, less than 0.4%, and most preferably, less than 0.3%, (or less than 0.22% or less than 0.1% or less than 0.05%) by weight of the wash composition of the invention. As to the non-sulfate-based ingredients, substantially free of also includes 0.0% (i.e., none) by weight of the ingredient in the wash composition of the invention. As to soap (e.g., sodium stearate or sodium oleate), the wash composition of this invention can have less than 2%, and preferably, less than 1%, and most preferably, less than 0.75% by weight soap, or less than 0.55%, or less than 0.25%, or from 0.001 to 0.24% or 0.0% (no soap) by weight soap based on total weight of the wash composition. With respect to all ingredients that the wash composition of the present invention may be substantially free of, it is within the scope of the invention to include from 0.001 to 0.045% by weight of such ingredient. In yet another preferred embodiment, the wash composition of the present invention comprises less than 35 ppm, and preferably, less than 25 ppm, and most preferably, less than 15 ppm dioxane or less than 2 ppm or less than 1 ppm dioxane or 0.0 ppm dioxane based on total weight of dioxane in the wash composition. In another embodiment, the wash composition of this invention may comprise from 0.00001 to 0.00005% by weight dioxane, like 1,4-dioxane.

The term comprising is meant to encompass the terms consisting essentially of and consisting of. For the avoidance of doubt, and for illustration, the wash composition of this invention comprising alpha olefin sulfonate sodium lauroyl isethionate and cocamidopropyl betaine is meant to include a composition consisting essentially of the same and a composition consisting of the same. All ranges defined herein are meant to include all ranges subsumed therein. Except in the operating and comparative examples, or where otherwise explicitly indicated, all numbers in this description indicating amounts or ratios of materials or conditions and/or physical properties of materials and/or use are to be understood as modified by the word “about”. The disclosure, as found herein, is to be considered to cover all embodiments as found in the claims as being multiply dependent upon each other irrespective of the fact that claims may be found without multiple dependency or redundancy.

### DETAILED DESCRIPTION OF THE INVENTION

As to the C₁₀₋₂₀ alpha olefin sulfonate suitable for use, the same is typically represented by the structure:

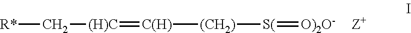

where R*is a C₆ to C₁₆ linear, branched, saturated or unsaturated hydrocarbon. When unsaturated R* preferably has 1 or 2, and most preferably, 1 double bond (i.e., each carbon atom of the bond being sp² hybridized). In an embodiment of the invention, R* is C₆ to C₁₆, and preferably, C₆ to C₁₄, and most preferably, C₈ to C₁₄, and even more preferably, C₁₀ to C₁₂. In an embodiment of the invention, at least 65%, and preferably at least 75%, and most preferably, from 80 to 100% or from 90 to 100% or from 95 to 100% or from 82 to 94% or from 82 to 90% or from 78 to 87% by weight of the alpha olefin sulfonate used is one where R* is C₁₀ to C₁₂ based on total weight of alpha olefin sulfonate in the wash composition. In even another embodiment, from 96 to 100% or from 92 to 99% or from 90 to 96% by weight of the alpha olefin sulfonate used is one where R* is C₁₀ to C₁₂ based on total weight of alpha olefin sulfonate in the wash composition.

Z⁺ is a counter ion that typically includes sodium, ammonium and/or potassium ions or a mixture thereof.

As to the co-anionic surfactant that may be used, acyl isethionate are suitable co-anionic surfactants. These include alkoxylated isethionate such as those described in Ilardi et al., U.S. Pat. No. 5,393,466, entitled “Fatty Acid Esters of Polyalkoxylated isethonic acid. The isethionate surfactants can include the reaction product of fatty acids esterified with isethionic acid and neutralized with a base like, for example, sodium or potassium hydroxide. The acyl isethionate surfactant can have the general formula:

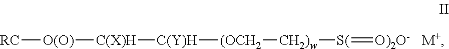

where R is a C₅ to C₁₉ saturated or unsaturated alkyl, w is an integer from 0 to 4 (1 to 4 for the alkoxylated option), X and Y are each independently hydrogen or an alkyl group having 1 to 4 carbons and M is a counter ion such as a Na⁺, K⁺, NH₄⁺, mixture thereof or the like.

It is also within the scope of the invention, when w is 0, for either the carbon alpha or beta to the sulfonate group to have one C₁₋₄ alkyl substitution in place of hydrogen, preferably a C₁₋₃ alkyl substitution and most preferably, a methyl group. It is also within the scope of the invention for R to have a degree of unsaturation, and typically, no more than 2, and preferably, no more than 1 double bond.

Illustrative examples of the isethionates suitable for use in the wash composition include sodium capryl isethionate, sodium caproylyl isethionate, sodium capryl methyl isethionate, sodium caproylyl methyl isethionate, sodium cocoyl isethionate, sodium cocoyl methyl isethionate, sodium lauroyl isethionate, sodium lauroyl methyl isethionate, potassium lauroyl isethionate, potassium lauroyl methyl isethionate, sodium oleoyl isethionate, sodium oleoyl methyl isethionate, sodium stearoyl isethionate, sodium stearoyl methyl isethionate, sodium myristoyl isethionate, sodium myristoyl methyl isethionate, sodium palmitoyl isethionate, sodium palmitoyl methyl isethionate, ammonium cocoyl isethionate, ammonium cocoyl methyl isethionate, or a mixture thereof. In an embodiment of the invention, isethionate selected for use comprises sodium lauroyl isethionate, sodium cocoyl isethionate or a mixture thereof. In another embodiment, the isethionate selected consists essentially of or consists of sodium lauroyl isethionate, sodium cocoyl isethionate or a mixture thereof. In an often-desired embodiment, the isethionate used is sodium lauroyl isethionate or sodium cocoyl isethionate.

Other co-anionics suitable for use with the alpha olefin sulfonates described include acyl taurates. Illustrative examples include sodium methyl lauroyl taurate, potassium methyl lauroyl taurate, sodium methyl myristoyl taurate, potassium methyl myristoyl taurate, ammonium methyl myristoyl taurate, sodium methyl cocoyl taurate, potassium methyl cocoyl taurate, ammonium methyl cocoyl taurate, sodium methyl oleoyl taurate, potassium methyl oleoyl taurate, ammonium methyl oleoyl taurate, sodium lauroyl taurate, potassium lauroyl taurate, ammonium myristoyl taurate, sodium cocoyl taurate, potassium oleoyl taurate, mixtures thereof or the like. In an embodiment of the invention, the taurate used is sodium methyl lauroyl taurate.

Even other co-anionic surfactants suitable for use with the alpha olefin sulfonates selected are acyl glycinates and salts thereof. Such glycinates include C₈ to C₂₀, and preferably, C₁₀ to C₁₈ and most preferably, C₁₂ to C₁₆ or C₁₂ to C₁₄ acyl glycinates. Illustrative and nonlimiting examples of the glycinates that may be included are sodium lauroyl glycinate, sodium myristoyl glycinate, sodium cocoyl glycinate, potassium lauroyl glycinate, sodium myristoyl glycinate, potassium cocoyl glycinate or a mixture thereof. In an embodiment of the invention, sodium cocoyl glycinate, potassium cocoyl glycinate or a mixture thereof are often preferred when acyl glycinates are used. In another embodiment, sodium or potassium lauroyl glycinate or both may also be used in the wash composition of the invention. In even another embodiment of the invention, the acyl portion of the glycinates that may be selected for use is preferably saturated but suitable to be unsaturated with no more than 2 double bonds, including conjugated double bonds.

Acyl glutamates (and salts thereof) may also optionally be included as co-anionic surfactant in the wash composition of the present invention. Glutamates such as C₈ to C₂₀, and preferably, C₁₀ to C₁₈ and most preferably, C₁₂ to C₁₆ or C₁₂ to C₁₄ acyl glutamates where the acyl portion is preferably saturated are suitable for optional use. As is the case with glycinates, the hydrophobic portion of the glutamates may be unsaturated with no more than 2 double bonds, including conjugated double bonds. Illustrative yet nonlimiting examples of the glutamates that may be used include sodium capryloyl glutamate, sodium lauroyl glutamate, sodium myristoyl glutamate, sodium cocoyl glutamate, sodium stearoyl glutamate, dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium olivoyl glutamate, sodium palmitoyl glutamate, sodium undecylenoyl glutamate, disodium cocoyl glutamate or a mixture thereof. In an embodiment of the invention, sodium lauroyl glutamate, sodium cocoyl glutamate, sodium stearoyl glutamate or a mixture thereof is preferred when glutamates are desired for optional use. In another embodiment, if a glutamate is used, sodium myristoyl and/or sodium cocoyl glutamate may often be desired.

Still other co-anionic surfactants suitable to be selected for optional use in the wash composition comprising alpha olefin sulfonate include alkyl sulfates, alkyl sulfonates, alkyl sulfosuccinates, alkyl ether sulfosuccinates, acyl sarcosinates or a mixture thereof (including any salts thereof).

As noted herein, the wash composition is, preferably, substantially free of surfactants that are sulfated (e.g., sulfate-based or comprising surfactants (e.g., sodium lauryl sulfate) and including those that are alkoxylated (e.g., sodium lauryl ether sulfate)). In an embodiment of the invention sulfated surfactants may be used, and again, preferably, at an amount of less than 0.6% by weight of the wash composition. If optionally included, the alkyl sulfates can include C₈-C₂₀ alkyl sulfates and/or alkoxylated versions such as sodium lauryl ether sulfate, sodium pareth ether sulfate or a mixture thereof where the ethoxylated portion (EO) is typically on average 1-3 EO groups. When selected, sodium lauryl sulfate, sodium lauryl ether sulfate, sodium pareth sulfate, ammonium lauryl sulfate or a mixture thereof are illustrative examples that may be included for for optional use as co-anionic.

As to additional sulfonates and their salts that may be selected for optional use as co-anionics, the same can include alkyl sulfonates, alkyl glyceryl ether sulfonates or a mixture thereof where the alkyl portion is typically an 8 to 18 or 10 to 16 hydrocarbon.

Suitable succinates (including their salts) that may optionally be included as co-anionics are disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, or a mixture thereof. For the avoidance of doubt, MIPA and MEA refer to monoisopropanolamine and monoethanolamine, sarcosinate or a mixture thereof. respectively.

The acyl sarcosinates suitable for optional use as co-anionic include those having a C₈-C₂₀ or C₁₀-C₁₈ or C₁₂-C₁₈ acyl group. Illustrative examples of the sarcosinates that may optionally be used include sodium lauroyl sarcosinate, sodium cocoyl sarcosinate, sodium stearoyl

In an embodiment of the invention, at least one co-anionic surfactant optionally selected is sodium lauryl sulfosuccinate, sodium myristoyl sulfosuccinate, sodium cocoyl sulfosuccinate, sodium stearoyl sulfosuccinate, sodium laureth sulfosuccinate, sodium pareth sulfosuccinate, disodium laureth sulfosuccinate, disodium lauryl sulfosuccinate, diethylhexyl sodium sulfosuccinate or a mixture thereof. In one embodiment of the invention, acyl sarcosinate makes up from 0.5 to 10% or from 1 to 8% or from 3 to 6% or 4 to 5% by weight of the total weight of anionic surfactant in the wash composition with sodium cocoyl sarcosinate, sodium lauroyl sarcosinate or a mixture thereof often being preferred when a sarcosinate is optionally used.

In yet another embodiment, optionally used co-anionic surfactant may include sodium methyl 2-sulfolaurate or disodium 2-sulfolaurate or both.

In a preferred embodiment of the invention, the co-anionic surfactant used is sodium lauroyl isethionate, sodium cocoyl isethionate or a mixture a mixture thereof. In still another preferred embodiment, the co-anionic surfactant used with the alpha olefin sulfonate is sodium lauroyl isethionate.

As described, the C₁₀₋₂₀ alpha olefin sulfonate makes up from 35 to 75%, and preferably, 40 to 70%, and most preferably, from 40 to 65% or from 45 to 60% or from 45 to 55% by weight of the total weight of the anionic surfactant in the wash composition. In even another preferred embodiment, the co-anionic surfactant is sodium lauroyl isethionate and the C₁₀₋₂₀ alpha olefin and sodium lauroyl isethionate are present at a weight ratio from 4:6 to 6:4 or from 4.2:5.8 to 5.8:4.2 or from 4.5:5.5 to 5.5:4.5 or from 1:1. Co-anionic surfactant, again, makes up from 1.2 to 4%, and preferably, from 1.5 to 3.5%, and most preferably, from 1.5 to 3% or from 1.6 to 2.6% or from 1.6 to 2.5% or from 1.7 to 2.2% by weight of the wash composition.

As to the C₁₀₋₂₀ alpha olefin sulfonate used, in an embodiment of the invention, from 90 to 100% or from 90 to 97% or from 92 to 99% or from 93 to 100% or from 95 to 100% or 97 to 100% or 100% by weight of the total weight of C₁₀₋₂₀ alpha olefin sulfonate is C₁₄₋₁₆ alpha olefin sulfonate.

As to the amphoteric surfactant suitable for use in the wash composition of the invention, the same can include betaines like lauryl betaine, lauryldimethyl betaine, coco betaine, cocamidopropyl betaine, laurylamidopropyl betaine, a mixture thereof or the like.

Still other amphoteric surfactants suitable for use include lauryl hydroxysultaine, cocamidopropyl hydroxysultaine or mixtures thereof. Even other amphoteric surfactants that may be used in the wash composition include alkyl amidopropyl hydroxysultaines including palmityl, stearyl and/or oleyl amidopropyl hydroxysultaine, and most preferably, palmityl amidopropyl hydroxysultaine. Other amphoteric surfactants suitable for use include behenyl betaine, capryl/capramidopropyl betaine, stearyl betaine, myristyl hydroxysultaine or a mixture thereof. In an embodiment of the invention, the amphoteric surfactant used comprises cocamidopropyl betaine, lauryl sulfobetaine, cocodimethyl carboxymethyl betaine, laurylhydroxy sulfobetaine, cocoamidopropylhydroxyl sulfobetaine, lauryl hydroxysultaine, a mixture thereof or the like. Additional amphoteric surfactants that may be used include C₁₀₋₁₈ alkylamidopropylamine N-oxides like laurylamidopropylamine N-oxide, C₁₀₋₁₈ alkyldimethylamine N-oxides like lauryldimethylamine N-oxide, C₁₀₋₁₈ alkylbetaines like laurylbetaine, or mixtures thereof.

In still another embodiment, the amphoteric surfactant used comprises lauryl sulfobetaine, lauryl hydroxysultaine or a mixture thereof.

Still other surfactants suitable for use having charge impacted by pH include cocamine oxide, lauramine oxide, myristamine oxide, palmitamine oxide, stearamine oxide, oleamine oxide, cocamidopropyl amine oxide, lauryl amidopropyl amine oxide, myristyl amidopropyl amine oxide, palmityl amidopropyl amine oxide, stearyl amidopropyl amine oxide, oleamidopropyl amine oxide or a mixture thereof. Additional optional examples of the amphoteric surfactants suitable for use include sodium lauroamphoacetate, sodium cocoamphoacetate, sodium lauroamphoacetate, sodium cocoamphoacetate, cocamphodipropionate or a mixture thereof.

In an embodiment of the invention the amphoteric selected can be at least 80%, and preferably, at least 85%, and most preferably, at least 90% (or 90 to 100% or 94 to 100% or 94 to 98% or 100%) by weight amine oxide whereby such amine oxide can have the formula: R^{x}—N⁺(R^{y})₂—O— where R^{x} is a C₈₋₂₀ alkyl, and preferably, a C₁₀₋₁₈ alkyl, and most preferably, a C₁₂₋₁₈ alkyl (or C₁₂₋₁₆ alkyl) and R^{y} is H or a C₁₋₆ alkyl or C₁₋₄ alkyl or ‘C₁₋₃ alkyl or C₁₋₂ alkyl or —CH₃. In another embodiment of the invention, amine oxide suitable for optional use is cocoyl amine oxide, lauramine oxide, myristamine oxide, palmitamine oxide, stearamine oxide, oleamine oxide or a mixture thereof. In still another embodiment of the invention, at least 85% or at least 90% or 90 to 100% or 100% by weight of the amphoteric surfactant used will be triglyceride derived and not fatty acid derived.

In yet another embodiment, the amphoteric surfactant used is from 92 to 100% or from 93 to 100% or from 93 to 98% or from 95 to 100% by weight cocamidopropyl hydroxysultaine, cocamidopropyl betaine or a mixture thereof based on total weight of amphoteric surfactant in the wash composition. In still another embodiment, the amphoteric surfactant used is 95 to 100% by weight cocamidopropyl betaine or 100% by weight cocamidopropyl betaine.

Amphoteric surfactant makes up from 3 to 10%, and preferably, from 4 to 8%, and most preferably, from 5 to 7.5% or from 5.5 to 7.5% or from 6 to 7.5% or from 6.5 to 7.25% or from 6.7 to 7.2% by weight of the wash composition.

In an embodiment of the invention other surfactants suitable for optional use may include imidazolines, sodium acyl amphoacetates, sodium acyl amphopropionates, disodium acyl amphodiacetates and disodium acyl amphodipropionates where the acyl (i.e., alkanoyl group) can comprise a C₇-C₁₈ alkyl portion and the same may make up from 0.001 to 1.2% or from 0.1 to 1% or from 0.1 to 0.5% by weight of the wash composition.

The wash composition of the present invention will comprise component comprising from 0.28 to 1.5%, and preferably, from 0.3 to 1.2%, and most preferably, from 0.3 to 1% or from 0.35 to 0.9% or from 0.38 to 0.8% or from 0.44 to 0.7% by weight of nonionic surfactant, C₁₀₋₂₀ fatty acid, C₁₀₋₂₀ fatty alcohol, C₁₀₋₂₀ fatty amide or a mixture thereof, with the proviso that if the C₁₀₋₂₀ fatty acid is included in the component, the fatty acid makes up from 25 to 50%, or from 30 to 45% or from 30 to 42% or from 30 to 40% by weight of the component. For the avoidance of doubt, component means at least one of nonionic surfactant, a C₁₀₋₂₀ fatty acid, C₁₀₋₂₀ fatty alcohol, C₁₀₋₂₀ fatty amide or any mixture thereof. In the case of C₁₀₋₂₀ fatty acid, component means a mixture comprising the same. In an embodiment of the invention, the component is 85 to 100% or from 90 to 100% or from 95 to 100% or from 95 to 98% by weight nonionic surfactant based on total weight of component. In another embodiment of the invention, the component is nonionic surfactant with 25 to 50% or from 30 to 45% or from 30 to 42% or from 30 to 40% by weight C₁₀₋₂₀ fatty acid based on total weight of component.

Nonionic surfactant which may be included can be the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic surfactant compounds are alkyl (C₆-C₂₂) phenols, ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine.

In an embodiment of the invention, nonionic surfactants selected can include fatty acid/alcohol ethoxylates having the following structures a) HOCH₂(CH₂)ₛ(CH₂CH₂O)ᵥ H or b) HOOC(CH₂)_{c}(CH₂CH₂O)_{d} H; where s and v are each independently an integer up to 18; and c and d are each independently an integer from 1 or greater. In an embodiment of the invention, s and v are each independently 6 to 18; c and d are each independently 1 to 30. Other options for nonionic surfactants include those having the formula HOOC(CH₂)ᵢ—CH═CH—(CH₂)ₖ(CH₂CH₂O)_{b} H, where i and k are each independently 5 to 15; and b is 5 to 50. In another embodiment of the invention, i and k are each independently 6 to 12; and b is 15 to 35.

The nonionic may also include a sugar amide, such as a polysaccharide amide. Specifically, the surfactant may be one of the lactobionamides described in U.S. Pat. No. 5,389,279 to Au et al., entitled “Compositions Comprising Nonionic Glycolipid Surfactants or it may be one of the sugar amides described in U.S. Pat. No. 5,009,814 to Kelkenberg, entitled “Use of N-Poly Hydroxyalkyl Fatty Acid Amides as Thickening Agents for Liquid Aqueous Surfactant Systems”.

In an embodiment of the invention and if used, the nonionic surfactant may comprise an alkyl polyglucoside represented as:

R¹—O—(S)ₙ, where R¹ is a C₆ to C₂₀ or C₈ to C₁₈ or C₁₀ to C₁₆ linear or branched alkyl and S is a saccharide group of 5 or 6 carbons such as glucose, xylose, lactose, fructose and/or mannose, and preferably, is glucose, and n represents the degree of polymerization and may have a value of from 1 to 12 or from 1 to 10, and most preferably, from 1.2 to 1.6 or from 1.3 to 1.5.

In yet another embodiment of the invention, the nonionic surfactant when used includes polysorbate (like polysorbate 20), cocamide monoethanolamine (cocamide MEA) or a mixture thereof. In even another embodiment, the nonionic surfactant when used is cocamide MEA. In still another embodiment of the invention, cocamide MEA makes up from 0.2 to 1.5%, and preferably, from 0.25 to 0.55%, and most preferably, from 0.3 to 0.5% or from 0.35 to 0.45% by weight of the wash composition.

Fatty acids like C₁₀ to C₂₀ or C₁₂ to C₁₈ or C₁₆ to C₁₈ fatty acids and amide and alcohol derivatives thereof (and any mixtures of the same) may be included in the wash composition of the invention. In an embodiment of the invention and when used, such components typically make up from 0.1 to 1% or from 0.15 to 0.8% or from 0.16 to 0.5% or from 0.19 to 0.35% or from 0.2 to 0.3% by weight of the wash composition. Preferred for use are fatty acids including palmitic acid, stearic acid, or a mixture thereof. In another embodiment of the invention, both palmitic acid and stearic acid are used at a weight ratio from 1:3 to 3:1 or from 1:2.5 to 2.5:1 or from 1:2.2 to 2.2:1 or from 1:1. In yet another embodiment, the fatty acid used is at least 50% by weight, and preferably, from 55 to 90%, and most preferably, from 60 to 100% or from 60 to 83% or from 90 to 100% or from 95 to 100% or 100% by weight stearic acid.

In a particular embodiment of the invention, the wash composition if free of (0.0% by weight) soap and sulfate-based surfactant. In another embodiment, the fatty acids like C₁₀ to C₂₀ or C₁₂ to C₁₈ or C₁₆ to C₁₈ fatty acids and amide and alcohol derivatives thereof or any mixtures thereof (i.e., fatty components, “FC”) and nonionic surfactant (“NS”) are included in the wash composition of the invention at a weight ratio where FC/NS is from 0.13 to 0.9, or from 0.2 to 0.9 or from 0.3 to 0.8 or from 0.4 to 0.7 or from 0.5 to 0.68. In still another embodiment, the fatty component is stearic acid, and the nonionic surfactant is cocamide MEA

Cationic surfactants may optionally be used and these include cetyltrimethylammonium bromide, cetyltrimethylammonium chloride, cetylpyridium chloride, behentrimonium chloride, stearalkonium chloride, lauryltrimethylammonium chloride, mixtures thereof or the like. If optionally used, such surfactants will often make up from 0.01 to less than 1%, or from 0.1 to 0.7% or from 0.2 to 0.5% by weight of the wash composition. In an embodiment of the invention, no (0.0%) cationic surfactant is used.

Inorganic salt is an optional ingredient to aid in composition consistency and water activity adjustment. Typical salts may be used like NaCl, KCl, MgCl₂, CaCl₂), mixtures thereof or the like. If used, such salt makes up less than 1.65%, and preferably, less than 1.6% and most preferably, from 0.05 to 1.55% or from 0.5 to 1.5%, or from 1 to 1.5% or from 1.1 to 1.45% by weight of the wash composition.
a. less than 1.65%, and preferably, less than 1.6%, and most preferably, from 0.05 to 1.55% or from 0.5 to 1.55% or from 1 to 1.5% or from 1.1 to 1.45% by weight salt;

Fatty acid sources for the surfactants described herein may, for example, include vegetable, soy, nut, jojoba, avocado, olive, seed oil (e.g., sunflower, linseed, rapeseed, moringa), fruit (e.g., mango, papaya, red berries, coconut), palm kernel and/or palm oil. In an embodiment of the invention, less than 5%, and preferably, less than 2%, and most preferably, less than 1% (or 0.0%) palm kernel oil is used to make surfactant included in the present invention. In another embodiment of the invention, from 0.001 to 70%, and preferably, from 0.01 to 60%, and most preferably, from 1 to 50% (or 2 to 39%) by weight of total surfactant used in the present invention is derived from palm oil and/or palm kernel oil. In even another embodiment, surfactant in the present invention is less than 5% by weight (or 0.0% by weight) palm kernel oil derived.

In even another embodiment of the invention, less than 95%, and preferably, less than 90%, and most preferably, less than 85% (or less than 80% or from 5 to 22% or from 6 to 20% or from 10 to 15%) by weight of the hydrophobic portion of surfactant used in the present invention is recovered from petroleum, palm oil, palm kernel oil and/or coconut oil. In still another embodiment, from 90 to 100% by weight of the hydrophobic portion of at least one surfactant used in the present invention is not recovered from petroleum, palm oil, palm kernel oil and/or coconut oil.

In a further embodiment of the invention, from 0.0 to 15% or from 0.01 to 12% or from 0.5 to 10% by weight of any of the surfactants used in the present invention may have hydrophobic portion with carbon recovered from purple carbon, and that is, carbon recovered from carbon dioxide or carbon monoxide or methane waste gas (or mixtures thereof) via biotechnology that utilizes microbial gas fermentation. It is also suitable in the invention to utilize carbon sources recovered from algal oils, microbial enhanced oil recovery processes and from plants like the oil recovered from Macauba pulp.

As to the peroxisome proliferator-activated receptor (PPAR) agonist suitable for optional use in, the wash composition of the present invention, if used, the wash composition will comprise from 0.001 to 1%, and preferably, from 0.01 to 0.8%, and most preferably, from 0.015 to 0.7% or from 0.018 to 0.7% or from 0.02 to 0.5% or from 0.03 to 0.35% or from 0.03 to 0.08% by weight of the PPAR agonist where such agonist is at least 65%, and preferably, at least 75%, and most preferably, from 80 to 100% or from 85 to 100% or from 90 to 100% or from 60 to 90% or from 90 to 98% or 100% by weight 12-hydroxystearic acid based on total weight of the PPAR agonist in the wash composition. Other PPAR agonists suitable for use with 12-hydroxystearic acid are preferably PPAR-alpha agonists including but not limited to 10-hydroxystearic acid, pertoselinic acid, conjugated linoleic acid, 12-hydroxy-9-cis-octadecanoic acid (ricinoleic acid), palmitoleic acid, stearolic acid or a mixture thereof.

Polyols (i.e., humectants) are suitable for optional use in the wash compositions of the present invention whereby the same are limited only to the extent that they are suitable for use in a consumer product. Illustrative and nonlimiting examples of the optional polyols suitable for use in the present invention include sorbitol, glycerol (glycerin), mannitol, xylitol, maltitol or mixtures thereof. In an embodiment of the invention, the polyol used is at least 50% by weight glycerol, based on total weight of the polyol used. In another embodiment of the invention, the polyol used is all glycerol (100% by weight). Polyol, when used, will typically make up from 0.5 to 12% by weight of the wash composition, and preferably, from 1 to 9% by weight of the wash composition, and most preferably, from 1 to 8% (or from 1 to 7% or 1 to 6.5% or from 1.5 to 5.5%) by weight of the wash composition.

Water, as noted, typically makes up from 70 to 92%, and preferably, from 72 to 88%, and most preferably, from 75 to 86% or from 78 to 85% by weight of the wash composition.

Adjusters suitable to modify/buffer the pH may optionally be used. Such pH adjusters include triethylamine, NaOH, KOH, H₂SO₄, HCl, C₆H₈O₇ (i.e., citric acid) or mixtures thereof. The pH adjusters are added at amounts to yield the desired final pH (as defined from 5.7 to 8). The pH values may be assessed with commercial instrumentation such as a pH meter made commercially available from Thermo Scientific®. adjuster, when used, is typically present at from 0.0001 to 0.7% or from 0.0002 to 0.3% or from 0.0005 to 0.22% by weight of the wash composition.

In an embodiment of the invention, the wash composition may optionally comprise polymeric suspending agent to contribute to, for example, product homogeneity, consumer skin appearance and sensory experience. Polymeric suspending agent, if used, typically makes up less than 0.6% by weight, and preferably, less than 0.4%, and most preferably, less than 0.3%, (or less than 0.22% or less than 0.1% or less than 0.05%) by weight of the wash composition of the invention.

Illustrative examples of optional suspending agent suitable for use include those which are a fermentation derived cellulose like Arbalon R-50® and Arbalon R-50® EV made available from Lubrizol. Such products comprise glycerin, cellulose and cellulose gum with 1.3 to 2.5 or 1.5 to 2.35 or 1.55 to 2.25 times more cellulose than cellulose gum. Glycerin typically makes up from 2.45 to 3.6 or from 2.55 to 3.5 or from 2.75 to 3.3 times more than the total amount of cellulose and cellulose gum. Often, the desired cellulose is one derived from a microbial fermentation process.

The wash composition may comprise thickening agent to additionally contribute to viscosity and sensory attributes of the composition. The same, if optionally used, typically makes up from 0.001 to 1.5% by weight, and preferably, from 0.06 to 0.4%, and most preferably, less than 0.3%, (or from 0.07 to 0.5% or less than 0.25% or less than 0.15% or less than 0.1%) by weight of the wash composition.

Illustrative examples of the polymeric thickening agent suitable for optional use include hydroxyethyl cellulose, hydroxypropyl methylcellulose, acrylates/steareth-20 methacrylate copolymer, PEG 120 methyl glucose trioleate (and) propanediol, acrylates/beheneth 25 methacrylate copolymer, xanthan gum, carbomer, acrylates/C10-30 alkyl acrylate crosspolymer, guar hydroxypropytrimonium chloride, carrageenan, xanthan gum/locust bean gum, xanthan gum/guar gum, cellulose gum, microcrystalline cellulose, cellulose gel, hydroxyethyl cellulose, hydroxypropyl cellulose, sodium carboxymethylcellulose, hydroxymethyl or carboxymethyl cellulose, methyl cellulose, ethyl cellulose, guar gum, gum karaya, gum tragacanth, gum Arabic, gum acacia, gum agar, xanthan gum, Versathix™ (PEG-150 Pentaerythrityl Tetrastearate (and) PPG-2 Hydroxyethyl cocamide and water), Aristoflex AVC (ammonium acryloyldimethyltaurate/VP copolymer), Carbopol® Ultrez 10 and 20 (crosslinked polyacrylic acid); alkaline soluble emulsion polymers such as Aculyn 28, Acuyln 22 or Carbopol Aqua SF1; cationic polymer such as modified polysaccharides including cationic guar available from Aventis under the trade name Jaguar C13S, Jaguar C14S, Jaguar C17, or Jaguar C16; cationic modified cellulose such as UCARE Polymer JR 30 or JR 40 from Amerchol, N-Hance 3000, N-Hance 3196, N-Hance GPX 215 or N-Hance GPX 196 from Ashland, Inc., synthetic cationic polymers such as MerQuat 100, MerQuat 280, Merquat 281 and Merquat 550 by Nalco, cationic starches, e.g., StaLok® 100, 200, 300 and 400 made by Galactasol 800 series by Henkel, Inc., Quadrosoft Um-200, Polyquaternium-24, Structure® ZEA (a hydroxypropyl modified corn starch) or Structure® XL, a cross-linked pregelatinized hydroxypropyl starch phosphate or a mixture thereof.

Oil may optionally be included in the wash composition of this invention. If used, oil could make up from 0.001 to 0.5% or from 0.005 to 0.3% or from 0.008 to 0.22% by weight of the wash composition. Oils that may be included are any of those suitable to contact skin, and preferably, those that are natural and sustainable. Illustrative examples of such oils include silicone oils and/or mineral oil, but those that are naturally sourced and sustainable like, arachis oil, castor oil, coconut oil, corn oil, cotton seed oil, olive oil, rapeseed oil, safflower seed oil, sesame seed oil, soybean oil, avocado oil, macadamia nut oil, argan oil, pomegranate oil, argan Moroccan oil, blueberry oil, raspberry oil, walnut oil, pecan oil, peanut oil, bayberry oil, mango seed oil, jojoba oil, hydrolyzed jojoba oil, mixtures thereof or the like are typically desired. If a silicone oil is optionally used, the same can include, for example, PEG-3 Dimethicone, PEG-8 Dimethicone, PEG-9 Dimethicone, PEG-10 Dimethicone, PEG-11 Methyl ether dimethicone, PEG-12 Dimethicone, PEG-14 Dimethicone, PEG-17 Dimethicone, PEG-32 Dimethicone mixtures thereof or the like. As noted, however, the wash composition of the present invention is preferably substantially free of silicone oil.

The oil used can have a droplet size from 20 nm to 10 microns or from 50 nm to 2 microns or from 30 nm to 1 micron. In an embodiment of the invention, oil is provided as a nanoemulsion with droplet sizes ranging from 50 to 800 nm or from 60 to 700 nm from 125 to 500 nm.

Optional water-soluble cosmetic agents suitable for use in the wash composition of the invention are limited only to the extent that they are capable of being used in a consumer product at the desired pH described.

Illustrative examples of the skin benefit agents suitable to include in the wash composition are acids, like amino acids, such as arginine, valine or histidine. Additional water-soluble benefit agents suitable for use include vitamin B₂, niacinamide (vitamin B₃), vitamin B₅ (Panthenol), vitamin B₆, folic acid (vitamin B₉), vitamin C, mixtures thereof or the like. Water soluble derivatives of such vitamins may also be employed. For instance, vitamin C derivatives such as ascorbyl tetraisopalmitate, magnesium ascorbyl phosphate and ascorbyl glycoside may be used alone or in combination with each other. Other water-soluble benefit agents suitable for use include 4-ethyl resorcinol, water soluble extracts of sage, aloe vera, green tea, grapeseed, thyme, chamomile, yarrow, cucumber, liquorice, rosemary extract or mixtures thereof. Water soluble sunscreens like ensulizole (phenylbenzimidizole sulfonic acid), disodium phenyl dibenzimidazole tetra sulfonate or mixtures thereof may also be included. Total amount of optional water-soluble benefit agents (including mixtures) when present in the wash composition may range from 0.001 to 10%, preferably from 0.001 to 5%, and most preferably, from 0.01 to 8% (or from 1 to 6% or from 1 to 3%) by weight of the wash composition.

It is also within the scope of the present invention to optionally include oil soluble cosmetic agents (i.e., non-water-soluble agents defined as less than 1.0 g of such agent (solute) can be dissolved in 100 ml of 25° C. water (solvent) at atmospheric pressure). The only limitation with respect to such oil soluble cosmetic agents are that the same are suitable for use in a consumer product.

Illustrative examples of the types of oil soluble cosmetic agents that may optionally be used in the wash composition of the invention include components like sunscreens and including but not limited to ethylhexylmethoxycinnamate, bis-ethyl hexyloxyphenol methoxyphenol triazine, 2-ethylhexyl-2-cyano-3,3-diphenyl-2-propanoic acid, drometrizole trisiloxane, 3,3,5-trimethyl cyclohexyl 2-hydroxybenzoate, 2-ethylhexyl-2hydroxybenzoate or a mixture thereof.

Other optional oil soluble cosmetic agents suitable for use include resorcinols like thiamidol (isobutylene thiazolyl resorcinol), 4-hexyl resorcinol, 4-phenylethyl resorcinol, 4-cyclopentyl resorcinol, 4-cyclohexyl resorcinol 4-isopropyl resorcinol or a mixture thereof. Also, 5-substituted resorcinols like 4-cyclohexyl-5-methylbenzene-1,3-diol, 4-isopropyl-5-methylbenzene-1,3-diol, mixtures thereof or the like may be used. The 5-substituted resorcinols, and their synthesis are described in commonly assigned U.S. Published Patent Application No. 2016/0000669A1.

Even other oil soluble cosmetic agents suitable for use include omega-3 fatty acids, omega-6 fatty acids, climbazole, farnesol, ursolic acid, myristic acid, geranyl geraniol, oleyl betaine, cocoyl hydroxyethyl imidazoline, vitamin E (and its derivatives including alpha-tocopherol acetate), hexanoyl sphingosine, terpineol, thymol, mixtures thereof or the like.

In an embodiment of the invention, the optional oil soluble cosmetic agent used is a retinoic acid precursor. In one embodiment of the invention, the retinoic acid precursor is retinol, retinal, retinyl propionate, retinyl palmitate, retinyl acetate or a mixture thereof. Retinyl propionate, retinyl palmitate and mixtures thereof are typically preferred when used.

When optional oil soluble cosmetic agent is used in the wash composition, such cosmetic agent typically makes up to 2% (or to 1.5%), and preferably, from 0.001 to 1.2%, and most preferably, from 0.05 to 0.85% by weight of the wash composition with the proviso that PPAR agonist makes up no more than 1% by weight of the wash composition.

Conventional preservatives can desirably be incorporated into the wash composition to protect against the growth of potentially harmful microorganisms. Cosmetic chemists are familiar with appropriate preservatives and routinely choose them to satisfy the preservative challenge test and to provide product stability. Suitable traditional preservatives that may be used include those traditionally found in consumer products. Preservatives suitable for preferred use are iodopropynyl butyl carbamate, phenoxyethanol, hydroxyacetophenone, ethylhexylglycerine, imidazolidinyl urea, sodium dehydroacetate, benzyl alcohol, sodium benzoate and mixtures thereof. Other preservatives suitable for use include sodium dehydroacetate, chlorophenesin and decylene glycol. The preservatives should be selected having regard for the use of the composition and possible incompatibilities between the preservatives and other ingredients included. Preservatives are preferably employed in amounts ranging from 0.01% to 2.3% (or 0.1 to 2% or 0.1 to 1.5%) by weight of the total weight of the wash composition. Also preferred is a preservative system with hydroxyacetophenone alone or in a mixture with other preservatives.

While suitable for use, but in an embodiment of the invention, the wash composition is substantially free of hydantoins, parabens and isothiazolinones.

In another embodiment, the preservative used is sodium benzoate (present at amounts from 0.2 to 1.6% or 0.9 to 1.4% or 1 to 1.3% by weight of the wash composition). In even another embodiment, 1,2-octanediol is used with preservative like sodium benzoate, phenoxyethanol, iodopropynyl butyl carbamate or a mixture of the same where the vicinal diol typically makes up from 0.5 to 1.5 or from 0.6 to 1.3 or from 0.7 to 1.2 times the amount of preservative used. Such diols are common emollients. Lipidated glycines are also suitable for use to boost preservative efficacy. If selected for use, such lipidated component may make up from 0.01 to 0.7% or from 0.1 to 0.6% or from 0.15 to 0.4% by weight of the wash composition. Often preferred lipidated components suitable for use are capryloyl glycine, undecylenoyl or a mixture thereof.

In even another embodiment, the wash composition made according to the invention may comprise thymol, terpineol, chloroxylenol, zinc pyrithione, piroctone olamine or a mixture thereof. In still another embodiment, the wash compositions may comprise N-acetyl methionine, N-acetyl cysteine (D, L isomers) or both. When selected for optional use, the same may make up alone or collectively from 0.01 to 3% or from 0.1 to 2% or from 0.2 to 1.5% or from 0.1 to 1% or from 0.1 to 0.6% by weight of the wash composition.

Fragrances, fixatives, chelators (like EDTA, citric acid, salicylic acid, triethanolamine), colorants (like caramel, beet juice, beta-carotene, vegetable juice or mixtures of the same), hydrated silica, alcohols (like polypropylene glycol-9), and exfoliants may optionally be included in the wash composition. Each of these substances may range from about 0.03 to about 4%, preferably between 0.01 and 3% (or from 0.05 to 1.2% or 0.07 to 1%) by weight of the total weight of the wash composition. To the extent exfoliants are used, those selected should be of small enough particle size so that they do not impede the performance of any packaging used to dispense the wash composition of the invention. In an embodiment of the invention, less than 0.7% or less than 0.4% or from 0.1 to 0.3% or 0.0% by weight EDTA is present in the composition.

In another embodiment of the invention, the wash composition will be substantially free of a sulfate-based surfactant and comprise at least one of botanical microspheres, thiamidol, oleyl adapalenate, hyaluronic acid, 10-hydroxystearic acid, 12-hydroxystearic acid, mandelic acid, lactic acid, caffeine, tretinoin ((all-E)-3,7-dimethyl-9-(2,6,6-trimethyl-1 cyclonexen-1-yl)-2,4,6,8-nonatetraenoic acid), epigallocatechin gallate, adapalene (6-[3-(1-adamantyl)-4-methoxyphenyl]naphthalene-2-carboxylic acid), 2-mecaptonicotinoyl glycine, salicylic acid, benzoyl peroxide, N-acetyl-DL-methionine, terpineol, thymol, glycerin, retinol, retinyl palmitate, retinyl propionate, 4-ethyl resorcinol, 4-hexyl resorcinol, or a mixture thereof. In another embodiment and when used, such ingredients make up (alone or collectively) from 0.0001 to 6%, or from 0.001 to 5%, or from 0.01 to 4% or from 0.01 to 3% or from 0.01 to 2% (or 0.1 to 1.5% or 0.1 to 1.25 or 0.1 to 4.3%) by weight of the wash composition.

When making the wash composition, the desired ingredients may be mixed with conventional apparatus under moderate shear and atmospheric conditions, with temperature being from 30 to 85° C. whereby shear continues until a homogeneous product is recovered.

The packaging for the wash composition typically is not limited if composition can be dispensed. In an embodiment of the invention, wash composition is sold in a pouch, bottle, jar, tube or canister. The packaging preferably is biodegradable and/or is prepared from recycled materials including post-consumer resins. Paper bottles are also suitable for use as are those that comprise bamboo. Other suitable formats include impregnating the wash composition in nonwovens like those having cotton, polyester or both or by providing the end use products in water soluble sachets such as those essentially consisting of polyvinyl alcohol.

When using the wash composition of the invention, the composition is often used with water at a composition to water weight ratio from 1:6 to 1:3 or from 1:5 to 1:4.5 or from 1:4.5 to 1:4, contacted with a surface, like skin, and rinsed off with water.

The Examples are provided to facilitate an understanding of the invention. The Examples are not intended to limit the scope of the claimed invention.

### Example I

A surfactant system consistent with the system used in the wash compositions made according to the invention, was made with the components listed in the Table I. The surfactant system also contained 1.2% preservative, 5% glycerin, 1% fragrance and pH adjusters, with the eight percents being by weight of the wash composition. All components were combined by mixing with moderate shear. Mixing temperature was stabilized between 55 to 80° C. and pressure was atmospheric. The resulting surfactant system was cooled to about 35° C. and pH was adjusted to 6.5. The percents shown in the Table and referred to in this Example are percents by weight. Viscosity was taken at 25° C. with a Discovery HR-2 Rheometer using sand blasted plates having a 1000-micron gap and a shear rate of 10-1 s. CAPB, AOS, SLI CMEA, and 12-HSA are cocamidopropyl betaine, alpha olefin sulfonate (C₁₄₋₁₆), sodium lauroyl isethionate, cocamide monoethanolamine and 12-hydroxystearic acid, respectively.

TABLE I

| | | | | | | | Viscosity | 25% | 25% |
|---|---|---|---|---|---|---|---|---|---|
| | | | | | | | For Full | Activity | Activity |
| | | | | | 12- | Stearic | Activity | Reduction | Reduction |
| Sample | CAPB | AOS | SLI | CMEA | HSA | Acid | (10 1/sec) | (10 1/sec) | (% Change) |
| 1 | 7 | 4 | 0 | 0.05 | 0.05 | 0.25 | 8,865 | 4,284 | −52 |
| 2 | 7 | 2 | 2 | 0 | 0 | 0 | 8,294 | 2,474 | −70 |
| 3 | 7 | 2 | 2 | 0.34 | 0.05 | 0.25 | 9,586 | 6,640 | −31 |
| 4 | 7 | 2 | 2 | 0.6 | 0.05 | 0 | 10.150 | 7,469 | −26 |
| 5 | 7 | 2 | 2 | 0 | 0.05 | 0.6 | 9,299 | 4,516 | −51 |
| 6 | 7 | 0 | 4 | 0.4 | 0.05 | 0.25 | 3,343 | 5,953 | 78 |

Surfactant systems were made consistent with the present invention, Samples 3 and 4. The compositions made according to the invention were stable, lathered well and rinsed off easily with water, displaying traditional characteristics enjoyed by trained panelists.

Surprisingly, when the compositions made according to the invention were reformulated with 25% by weight less total solid (in the example active surfactant) to mimic ambiguities at production due to fluctuations in ingredient supply, the compositions having reduced surfactant and made according to the present invention did not display a viscosity change or difference of more than 31%, a delta not discernible to the trained panelists. Compositions that failed, not made consistent with the invention, displayed a discernible viscosity change of 36% or more when they were made with 25% less total solid.

### Example II

The formula provided in Table II was made as complete end use formula to include the systems depicted and made in Example I

TABLE II

| Ingredient Name | % by Weight |
|---|---|
| Deionized Water | Balance |
| Thickener | 0.1 |
| Sodium C14-C16 Olefin Sulfonate | 2 |
| Sodium Lauroyl Isethionate | 2 |
| Cocamidopropyl Betaine | 7 |
| Cocamide MEA | 0.3 |
| Stearic Acid C18 | 0.3 |
| 12-Hydroxystearic acid | 0.05 |
| Glycerin | 5 |
| oil | 0.01 |
| Inorganic Thickener | 0.9 |
| Exfoliant | 0.13 |
| Preservative | 1.2 |
| pH Buffer | 0.002 |
| Salt | 0.001 |
| Alcohol | 0.001 |
| Fragrance | 0.9 |

What is claimed is:

1. A wash composition comprising:
a. from 1.2 to 4% by weight of C₁₀₋₂₀ alpha olefin sulfonate;
b. from 1.2 to 4% by weight of a co-anionic surfactant;
c. a component comprising from 0.28 to 1.5% by weight of a nonionic surfactant, a C₁₀₋₂₀ fatty acid, C₁₀₋₂₀ fatty alcohol, C₁₀₋₂₀ fatty amide or a mixture thereof with the proviso that if the C₁₀₋₂₀ fatty acid is included in the component, the fatty acid makes up from 25 to 50%, by weight of the component;
d. from 3 to 10% by weight amphoteric surfactant;
e. optionally, from 0.001 to 1% by weight of a peroxisome proliferator-activated receptor (PPAR) agonist, the PPAR agonist comprising at least 65% by weight 12-hydroxystearic acid based on total weight of the PPAR agonist;
f. less than 1.65% by weight salt
g. from 68 to 90% by weight water,
wherein:
i) the composition is substantially free of sulfate-based surfactant;
i) the composition is isotropic;
iii) the C₁₀₋₂₀ alpha olefin sulfonate makes up from 35 to 75%, and preferably, 40 to 70%, and most preferably, from 40 to 65% or from 45 to 60%, or from 45 to 55% by weight of the total weight of the anionic surfactant in the wash composition; and
iv) the composition has a pH from 5.7 to 8, and preferably, from 6 to 7.5, and most preferably, from 6.1 to 7.2 or from 6.1 to 6.9 or from 6.2 to 6.8.

2. The wash composition according to claim 1 wherein the alpha olefin sulfonate is a C₁₄₋₁₆ alpha olefin sulfonate.

3. The wash composition according to claim 1 wherein the co-surfactant is sodium lauroyl isethionate, sodium methyl lauroyl taurate or a mixture thereof.

4. The wash composition according to claim 1 wherein the composition further comprises a preservative.

5. The wash composition according to claim 4 wherein the preservative is sodium benzoate present at 0.2 to 1.6% by weight of the composition.

6. The wash composition according to claim 1 wherein the PPAR agonist is present in the wash composition.

7. The wash composition according to claim 1 wherein the composition further comprises 10-hydroxystearic acid, pertoselinic acid, conjugated linoleic acid, 12-hydroxy-9-cis-octadecanoic acid, palmitoleic acid, stearolic acid or a mixture thereof.

8. The wash composition according to claim 1 wherein the wash composition is substantially free of a sulfate-based surfactant, paraben, hydantoin, isothiazolinone, phthalate, soap, dioxane and silicone oil.

9. The wash composition according to claim 1 wherein the wash composition further comprises thiamidol, oleyl adapalenate, hyaluronic acid, 10-hydroxystearic acid, mandelic acid, lactic acid, caffeine, tretinoin, epigallocatechin gallate, adapalene, 2-mecaptonicotinoyl glycine, salicylic acid, benzoyl peroxide, terpineol, thymol, glycerin, retinol, retinyl palmitate, retinyl propionate, 4-ethyl resorcinol, 4-hexyl resorcinol, or a mixture thereof.

10. The wash composition according to claim 1 wherein the component comprises fatty acid that is stearic acid, the nonionic that is cocamide MEA.

11. A method for washing a surface comprising, in no particularly order, the steps of contacting a surface with the wash composition according to claim 1 and water, and rinsing the composition off with water.

12. The method for washing a surface according to claim 11 wherein the surface is skin or hair.
